(19) United States
(12) Patent Application Publication
(10) Pub. No.: US 2015/0309025 A1
(43) Pub. Date: Oct. 29, 2015

(54) PROCESS AND MACHINE FOR AUTOMATED AGGLUTINATION ASSAYS

(71) Applicant: Gold Standard Diagnostics, Davis, CA (US)

(72) Inventors: Peter Van Praet, Haasrode (BE); Jennifer Roth, Sacramento, CA (US); John Griffiths, Davis, CA (US)

(21) Appl. No.: 14/334,316

(22) Filed: Jul. 17, 2014

Related U.S. Application Data
(60) Provisional application No. 61/847,469, filed on Jul. 17, 2013.

(51) Int. Cl. G01N 33/571

(52) U.S. Cl. CPC G01N 33/571

(57) ABSTRACT

The machine is configured to perform an automated rapid plasma reagent (RPR) agglutination test or other agglutination test. The machine includes a sample rack with multiple sample locations thereon and a reagent rack for storing of reagent. A shaker assembly supports at least one microtiter plate or other well supporting structure thereon with a plurality of wells in the plate. An automated syringe or other aspirator and dispenser accesses samples and reagent and deposits them within wells of the microtiter plate. The shaker assembly shakes multiple samples within the wells of the microtiter plate according to the RPR or other agglutination test. Finally, a camera photographs the wells of the plate, preferably from above with a light source below and the plate at least partially transparent, to evaluate whether the specimen is reactive or non-reactive. Test results and photographic evidence of the test results are preferably archived within a database.

### CROSS-REFERENCE TO RELATED APPLICATIONS

This application claims benefit under Title 35, United States Code §119(e) of U.S. Provisional Application No. 61/847,469 filed on Jul. 17, 2013.

### FIELD OF THE INVENTION

The following invention relates to machines and automated processes for performing automated agglutination assays, such as those used in a rapid plasma reagin (RPR) test. More particularly, this invention relates to machines and processes for automated conducting of assays such as a BD Macro-Vue RPR test, such as that utilized to detect syphilis.

### BACKGROUND OF THE INVENTION

Etiological agents responsible for various infections and other diseases, such as syphilis can be detected in a test referred to as a rapid plasma reagin (RPR) test. One specific such assay is known as a BD Macro-Vue RPR test provided by Becton Dickinson and Company of Franklin Lakes, New Jersey. The RPR test is a non-treponemal flocculation test that is used to detect and quantify reagin, an antibody present in serum or plasma as a screen test for syphilis. The etiological agent responsible for syphilis produces at least two kinds of antibodies in human infections. The treponemal antibodies can be detected by florescent treponemal antibody-absorption (FTA-ABS) test whereas the reagin antibody is detected by the RPR antigen test. In the presence of the reagin antibody and the reactive sample, the RPR antigen preparation will produce flocculation consisting of black clumps against the white background of a test card. By contrast, non-reactive samples will yield an even light gray homogenous suspension.

The RPR test known in the prior art is performed upon EDTA plasma and unheated or heated serum. The specimen should be free of bacterial contamination and haemolysis. A reagent is also utilized in the test. One such reagent is an RPR carbon antigen formed of 0.003 percent cardiolipin, 0.020-0.022 percent lecithin, 0.09 percent cholesterol, 0.0125 M EDTA, 0.1 MNa₂HPO₄, 0.01 MKH₂PO₄, 0.1 percent thimerosal, 0.0188 percent charcoal and ten percent choline chloride.

In performing the test the specimen and the reagent are combined together on a test card, such as by applying a drop of each onto the test card. The sample and antigen reagent are not mixed. Rather, they are put onto an automatic rotator, preferably under a humidity cover, with the rotator rotating the combination of the sample and reagent at 100 rpm for eight minutes. Following rotation, a brief hand rotation and tilting of card (three to four times) should be made to aid in differentiating non-reactive from minimally reactive results. Results are then read by studying the combination of the sample and the reagent. A non-reactive sample will have no clumping of the carbon particles in the reagent or very slight roughness, with a smooth gray overall appearance. If the sample is reactive, presence of large aggregates of carbon particles will be visually detected and usually against a clear background. A reactive specimen is considered to have undergone agglutination. In a more detailed variation of the test for more quantitative results, the sample is diluted two to one, four to one, eight to one, sixteen to one, etc. and the reagent is added and after rotation the sample is read for agglutination. In such a test those specimens which are non-reactive can be distinguished from those which are reactive and also minimally reactive specimens can be identified where there is a presence of small or fine aggregates of carbon particles.

Such a test involves combining a sample of a prepared blood product with an appropriate reagent that includes carbon (e.g. charcoal) particles therein. The reagent may or may not react with the specimen by undergoing flocculation. If the carbon particles become trapped in the flocculation and appear agglutinated or as black clumps against a light background, the specimen is considered to be reactive with the reagent. If the reagent maintains a uniform light gray color with even particle distribution and no clumping, it is indicative of a non-reactive specimen.

Known RPR tests, are currently known to be performed manually and to involve a variety of steps where the potential for human error or variation in manual performance of the test can result in less reliable results. Also, the test is significantly time intensive even when properly performed, requiring significant amounts of time expenditure by well trained practitioners. Accordingly, a need exists to automate the RPR test to more rapidly and reliably conduct tests with fewer skilled operator hours being required. Furthermore, it is desirable to have test results archived in a variety of different ways for later analysis and for verification of test results. By automating the RPR test, an opportunity is presented for high quality archiving of large numbers of assays for efficient and reliable management of test results from RPR tests or agglutination assays.

### SUMMARY OF THE INVENTION

With this invention a process is provided for automated agglutination assay performance for use in tests such as an RPR test, as well as a robotic analyzer for automating the performance of the processes of this invention. The process generally involves a series of steps which can be performed in sequence by the machine of this invention or a related machine for multiple samples. The sequence for one sample can overlap with the sequence for other samples in the same machine to maximize efficacy.

In one embodiment the steps are generally defined as loading samples/specimens into a sample rack of a machine, loading reagent into a reagent rack of the machine, loading a microtiter plate (or other structure with one or more wells or other test locations therein) onto a shaker assembly of the machine, using an automated microsyringe (or other aspirator and dispenser) to gather a sample from the sample rack and reagent from the reagent rack and deposit the combined sample and reagent in a well of the microtiter plate, shaking the microtiter plate for a predetermined amount of time, detect agglutination such as by photographing the well of the microtiter plate, reading the photograph for a result (reactive or non-reactive) and archiving the photograph and/or result within a database.

One machine capable of housing a plurality of samples, the reagent, and also to carry the shaker assembly and a carriage for automated motion of the microsyringe and camera is disclosed herein in a preferred embodiment. The machine has an overall housing with a lower region, a mid-region and an upper region. The lower region includes at least one sample rack with multiple locations therein. Most preferably, this sample rack is a “smart rack” which can carry test tubes or other containers of samples which can themselves have a bar code thereon and a scanner is built into the machine so that the samples are intelligently known by the machine to be positioned wherever they are placed within the rack. The reagent rack is also preferably in this lower region of the enclosure of the machine.

A midlevel of the machine preferably supports a shaker assembly thereon. This shaker assembly preferably only has half of a depth of the overall enclosure and can move forward and rearward. In this way, all of the sample racks and reagent rack locations can be accessed by moving the shaker assembly out of the way (either forward or backward). The shaker assembly is configured to support at least one microtiter plate thereon with each microtiter plate including a plurality of wells thereon. The shaker assembly is also configured with a shaker motor which can shake the microtiter plates upon the shaker assembly.

An upper portion of the enclosure has a carriage therein which preferably supports both an automated syringe, such as a microsyringe, and a camera. The carriage is configured to allow the microsyringe and camera to move both laterally and forwardly and rearwardly to access each of the samples of the sample rack, each of the wells of the microtiter plates on the shaker assembly and each of the reagent containers within the reagent rack. Appropriate robotics cooperate with the carriage and the shaker assembly to cause the microsyringe to move where required to gather a sample and reagent, and deposit them on an appropriate one of the wells within one of the microtiter plates. The robotic equipment then causes one or more combined specimen and reagent combinations to be shaken by the shaker assembly for a predetermined amount of time. A camera is then carried by the carriage to appropriate locations for photographing the wells of the microtiter plate. In a preferred embodiment the shaker assembly is configured with a diffuser beneath an at least partially transparent microtiter plate and with at least one LED light source below the diffuser, so that the wells of the microtiter plate are backlit during the photographing process.

### OBJECTS OF THE INVENTION

Accordingly, a primary object of the present invention is to provide a process for automating an RPR agglutination test.

Another object of the present invention is to perform an RPR agglutination test in a reliable fashion.

Another object of the present invention is to perform an RPR agglutination test with more rapid throughput of multiple samples.

Another object of the present invention is to provide an RPR agglutination test which records results of the test in a manner allowing review of both test results and underlying photographic data upon which test result conclusions are based.

Another object of the present invention is to provide a machine which automates an RPR agglutination test.

Another object of the present invention is to provide a machine which accurately performs multiple RPR agglutination tests on multiple separate samples accurately and efficiently.

Another object of the present invention is to provide a machine and process for performing an RPR agglutination test which minimizes the potential for human error in performing the test.

Other further objects of the present invention will become apparent from a careful reading of the included drawing figures, the claims and detailed description of the invention.

### BRIEF DESCRIPTION OF THE DRAWINGS

FIG. 1 is a perspective view of a machine according to this invention which can perform an RPR or other agglutination test on multiple samples in an automated fashion.

FIG. 2 is a perspective view of a shaker assembly within a midlevel of the machine of FIG. 1 and with a single microtiter plate loaded thereon.

FIG. 3 is a perspective view of that which is shown in FIG. 2 but with four microtiter plates located thereon and showing rails upon which the shaker assembly is carried.

FIG. 4 is a full sectional side elevation view of the shaker assembly of FIGS. 2 and 3 revealing interior details thereof.

FIG. 5 is a flow chart depicting the steps in the automated agglutination test of this invention.

### DESCRIPTION OF THE PREFERRED EMBODIMENT

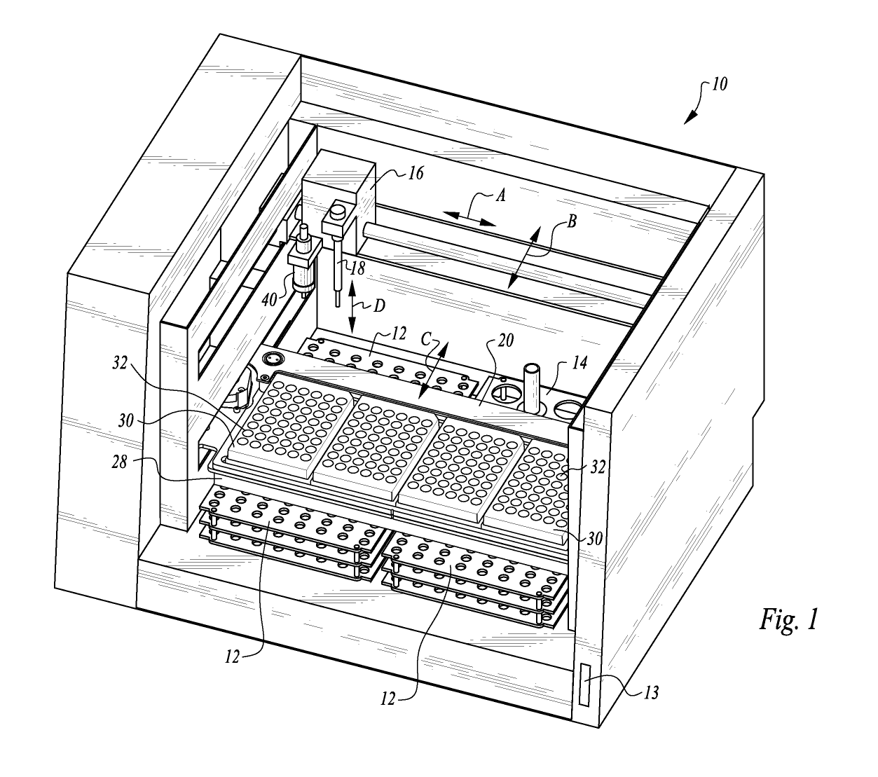

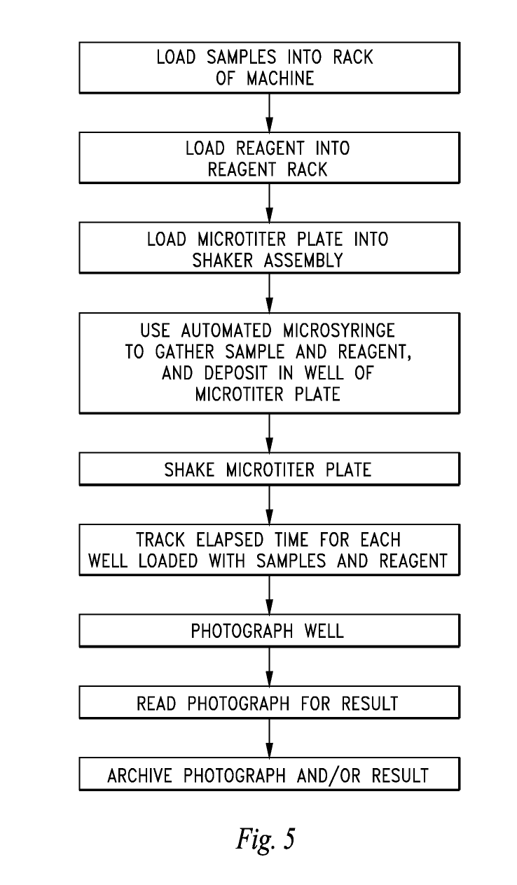

Referring to the drawings, wherein like reference numerals represent like parts throughout the various drawing figures, reference numeral 10 (FIG. 1) is directed to a machine for implementing an automated agglutination test process (FIG. 5) of this invention. The machine 10 can be loaded with samples, such as within the sample rack 12 and has multiple wells 32 upon a microtiter plate 30 where samples can be combined with a reagent and shaken a specified time according to the particular agglutination test protocol, such as for an RPR agglutination test. A camera 40 takes photographs of wells 32 within the microtiter 30 to record results of the test.

In essence, and with particular reference to FIG. 1, basic details of the machine 10 are described. The machine 10 includes an enclosure with an interior generally divided into a lower portion, a mid-portion and an upper portion. The overall enclosure can be similar to that of a robotic analyzer for providing a variety of different assays and other tests in an at least partially automated fashion. A lower portion of the interior of the enclosure has at least one sample rack 12 therein. A reagent rack is also located within this lower portion of the interior of the enclosure. A midlevel of the interior of the enclosure has a shaker assembly supported therein. The shaker assembly supports at least one microtiter plate 30 thereon in a manner which facilitates shaking of the entire microtiter plate. An upper portion of the interior of the enclosure has an upper carriage therein. The upper carriage can move both laterally and forwardly and rearwardly. The upper carriage carries an automated syringe, such as a micro syringe, and a camera so that the microsyringe and the camera can access each of the wells 32 of the microtiter plate 30 and so that the microsyringe 18 can access each of the locations in the sample rack 12 and the reagent rack 14.

The machine 10 is programmed to manipulate samples and reagents through the microsyringe and the microtiter plate upon the shaker assembly to perform the agglutination test. The test is then read by the camera 40 and results for each sample, along with pictures taken from the camera can be archived within a database which is correlated with information relating to the sample and other details of the test.

More specifically, and with reference primarily to FIG. 1, the particular details of the automated RPR agglutination test as conducted by the machine 10 are described, according to this preferred embodiment disclosed herein. With adjustment, such as using different reagent and/or different shaking procedures, other agglutination tests can similarly be performed. Initially, samples to be tested are loaded into the sample rack 12 of the machine 10. This sample rack 12 preferably has multiple locations where test tubes or similar structures containing a sample can be placed. Most preferably, the machine 10 includes a barcode scanner 13 thereon and tubes or other structures containing samples can have a barcode thereon so that when the sample is loaded into the rack 12 of the machine 10, the space in the rack 12 which has been loaded with the sample has been correlated with data associated with the barcode on the sample container. The user thus does not need to keep track of which space in the rack 12 has been loaded with each sample. One such rack 12 suitable for this invention is described in U.S. Published Patent Application No. 2012/0178170, incorporated herein by reference.

A reagent rack 14 is also provided into which reagent liquid is placed. Details about one appropriate reagent are described above in the Background. Preferably, this reagent rack 14 also includes a cleaning reservoir containing a cleaning solution for cleaning of the microsyringe or other fluid transfer device in between fluid transfer procedures.

Because the reagent typically has carbon particles within the liquid reagent which have a tendency to settle and detrimentally affect the quality of the reagent taken up by the microsyringe during operation of the procedure of this invention, the reagent rack 14 preferably includes a stirrer associated therewith to keep the carbon particles in suspension. In one embodiment this stirrer is a magnetic stirrer. Such a stirrer can have an impeller contained within the reagent fluid itself and which is caused to spin and keep the reagent stirred by an adjacent rotating magnetic field such as that provided by an electromagnet beneath the reagent rack 14. Other forms of stirrers could be utilized including mechanical stirrers or stirrers which repeatedly aspirate and dispense reagent sufficiently rapidly to keep the carbon products within the reagent suspended.

The microtiter plate 30 (or other well supporting structure) is loaded onto the shaker assembly 20. The microtiter plate 30 includes a plurality of wells 32 or other spaces (or at least one space in a simplest embodiment) thereon which can receive samples and reagents. The shaker assembly 20 is configured so that it can shake such as by rotating the microtiter plate 30 at 100 RPMs with an amplitude of about five millimeters. Preferably, multiple wells 32 are located on the microtiter plate 30 so that multiple samples and reagents can undergo reactions on the common microtiter plate 30 and be shaken by the common shaker assembly 20.

An automated microsyringe 18 or other fluid aspirator and dispenser gathers a sample and reagent and deposits the combined sample and reagent onto a well 32 or space on the microtiter plate 30. Most preferably, this microsyringe 32 is carried upon an upper carriage 16 which can move over the various different samples on the sample rack 12 and can also move over the reagent rack 14. The microsyringe 18 will typically first gather a predefined quantity of reagent and then further gather a predefined amount of sample and then carry both the reagent and sample to a well 32 on the microtiter plate 30 for dispensing thereon. The microsyringe 18 or other fluid transfer device could then pass to a cleaning reservoir such as adjacent the reagent rack 14 to undergo a cleaning procedure and then can gather further reagent and sample from another location on the sample rack 12 and deposit them onto another well 32 on the microtiter plate 30 located on the shaker assembly 20; and so on essentially ad infinitum.

The microtiter plate 30 undergoes shaking through action of the shaker assembly 20. The elapsed time is also tracked for each well 32 that has been loaded with a sample and reagent. After an amount of elapsed time and shaking called for by the testing protocol has been achieved, a camera 40 is aligned with the well 32 of the microtiter plate 30 which has the sample and reagent thereon and a photograph is taken of the well 32. The shaker assembly 20 preferably includes a light diffuser plate 27 beneath the microtiter plate 30 and the microtiter plate 30 (or other well support structure) is preferably formed of a transparent or translucent material to allow light to travel up through the microtiter plate 30.

An LED board 29 with a plurality of light emitting diodes surface mounted on a printed circuit board is preferably contained within the shaker assembly 20 beneath the diffuser 27 and supplied with power so that light from the LED board 29 shines up through the diffuser 27 and up through the microtiter plate 30. A backlit photograph is thus taken by the camera 40 from above looking down on the well 32 of the microtiter plate 30.

The LEDs are selected to minimize heat generation and are well ventilated to keep heat from transferring up to the microtiter plate 30. A fan can also optionally be provided to keep temperature substantially constant and at a desired temperature.

The photograph taken by the camera 40 is read to determine whether agglutination has occurred or not, and whether a positive or negative test is to be indicated. In one embodiment the reading of the photograph occurs by a trained professional. In other embodiments software might be employed to evaluate the image taken by the camera 40 with the software program automatically determining whether or not a positive test is indicated.

The photograph and/or the result of reading the photograph can be archived in a database also containing information such as that associated with the barcode on the sample container, and other information such as the date of the test, lot numbers or the reagent, and any other pertinent information (e.g. temperature at time of test, humidity at time of test, atmospheric pressure at time of test, etc.).

After all the wells 32 in the microtiter plate 30 have been utilized, the microtiter plate 30 can be disposed of or potentially sanitized for reuse. In addition to the basic procedure identified above, with many RPR tests it is desirable to re-perform the tests multiple times at different reagent and/or sample dilution levels. This series of titers can be selected as desired for the parameters of the RPR tests to be conducted. In one embodiment, dilution of the reagent can occur by including a diluting solution in the reagent rack 14 and having the microsyringe 18 or other automated fluid transport device take up both a predetermined amount of reagent and a predetermined amount of diluting solution, and then gathering a predetermined amount of sample from the sample location on the sample rack before transferring the combined gathered liquids to a well 32 or other location on the microtiter plate 30.

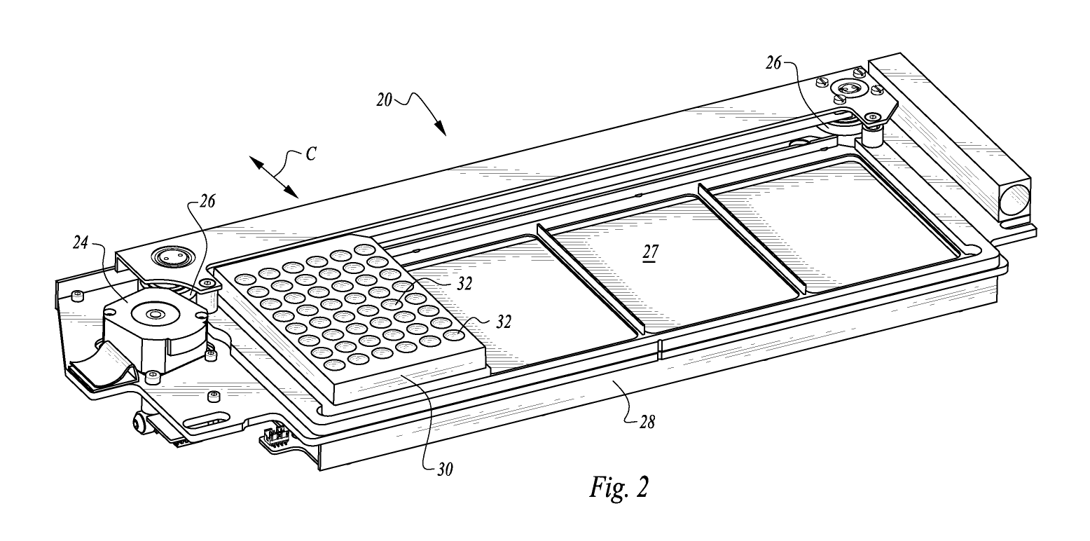

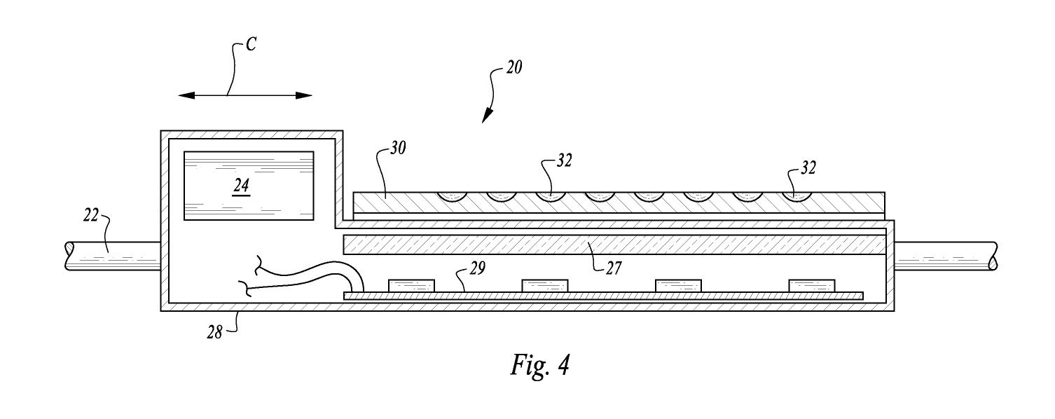

With particular reference to FIGS. 1-4, further details of the machine 10 for performing the process of this invention are described according to one embodiment of this invention. The machine 10 is generally in the form of an enclosure which includes a lower level, midlevel or upper level. The sample racks 12 preferably reside at the lower level and also a reagent rack 14. The midlevel of the enclosure is configured with the shaker assembly 20 riding upon rails 22 to allow the shaker assembly 20 to move forward and backward within the enclosure at a midlevel above the sample racks 12 and the reagent rack 14. An upper level of the enclosure includes the upper carriage 16 which rides on a bar which spans the enclosure laterally and can also be carried forwardly and rearwardly within the enclosure. A cover can isolate the entire enclosure, which pivots on a rear hinge. Such a cover is omitted in FIG. 1 to most clearly show the interior details of the machine 10.

The camera 40 and the automated microsyringe 18 (or other fluid aspirator and dispenser) are carried upon the upper carriage 16 in a manner which allows the camera 40 and automated microsyringe 18 to move laterally upon the upper carriage (along arrow A of FIG. 1). The upper carriage itself can move front to back (along arrow B of FIG. 1). Both the camera 40 and automated microsyringe 18 thus have access to be placed directly over each of the wells 32 within the microtiter plates 30 located upon the shaker assembly 20 and the microsyringe has access to each of the sample containers within the sample rack 12 and each of the reagent containers within the reagent rack 14.

The shaker assembly 20 is configured so that it can move front to back (along arrow C of FIG. 1). The automated microsyringe is configured so that it can move up and down (along arrow D) such as to access samples located within the sample rack 12 or to access reagents or diluting agents contained within the reagent rack 14.

The enclosure preferably includes the barcode scanner 13 built thereinto so that when the sample rack 12 is configured to hold tubes of samples, a barcode sticker can be placed on an exterior of the tube or other sample holder and the tube can first have its associated barcode scanned by the barcode scanner 13 before the tube is placed into one of the locations within the sample rack 12. The sample rack 12 is “intelligent” in that it can recognize when a tube has been placed therein. The rack 12 thus associates the recently loaded location in the rack 12 with the most recently scanned barcode so that a user does not need to place the test tube into a particular location, but the system automatically records where the sample test tube has been located within the sample rack 12. In this manner, an operator can load samples into the sample rack 12 by first passing tubes containing samples past the barcode scanner 13 and then placing them into a vacant location on the sample rack 12.

During this procedure, the shaker assembly 20 is typically located at a rear of the enclosure. If a large number of samples are being stored on the sample rack, 12 a rear lower portion of the enclosure can have portions of the sample rack 12 located there and the shaker assembly 20 can move to a forward location so that an operator can access locations within the sample rack 12 rear area. Reagent materials are supplied into appropriate reagent locations on the reagent rack 14 when the shaker assembly 20 is in a forward position (moving forward along arrow C of FIG. 1) so that the reagent rack 14 can be accessed. Software and/or sensors can be employed to prevent collisions, such as between the microsyringe 18 and the shaker assembly 20.

At least one microtiter plate 30 is loaded onto the shaker assembly 20. The shaker assembly 20 is configured so that four “6×8” microtiter plates 30 can be provided thereon which each include forty-eight wells 32. A magnetic stirrer or other stirrer associated with the reagent rack 14 is activated to keep carbon particles within the reagent in suspension. The machine 10 is now ready to automatically perform the RPR assay test according to particular design protocols for the test to be conducted.

First, the upper carriage 16 is positioned so that the automated microsyringe 18 can access the reagent container on the reagent rack 14. Any dilution fluid is also gathered from the reagent rack 14. Next, the automated microsyringe 18 moves upon the upper carriage 16 and the upper carriage 16 moves itself (along arrows A and B of FIG. 1) to place the automated microsyringe 18 over the appropriate location on the sample rack 12 to gather a portion of one of the samples into the microsyringe 18. A portion of the sample is then aspirated. The microsyringe 18 is then elevated (along arrow D of FIG. 1) and through a combination of movement of the upper carriage 16 and the shaker assembly 20 (along arrows A, B and C) the automated microsyringe is placed over one of the wells 32 on one of the microtiter plates 30 resting on the shaker assembly 20. The automated microsyringe 18 then is moved down over the well 32 (along arrow D of FIG. 1) and caused to dispense the sample, reagent and any diluting agent into the well 32.

A shaker motor 24 is activated and the shaker assembly 20 is caused to shake the microtiter plate 30. The shaker motor 24 is preferably coupled to an eccentric 26 weight or weights, or coupled to a belt that is unbalanced or other known shaker elements are utilized to perform the desired shaking. The machine 10 keeps track of the time that the reagent and sample came into contact or were dispensed into the well 32. Multiple times for multiple wells 32 can be simultaneously tracked. The shaker assembly 20, and upper carriage 16 and automated microsyringe 18 can repeat the above process to gather further reagent and further sample, typically after a self-cleaning procedure for the microsyringe 18 is conducted. In this way, a second sample and reagent combination can be dispensed onto a second well 32 on the microtiter plate 30.

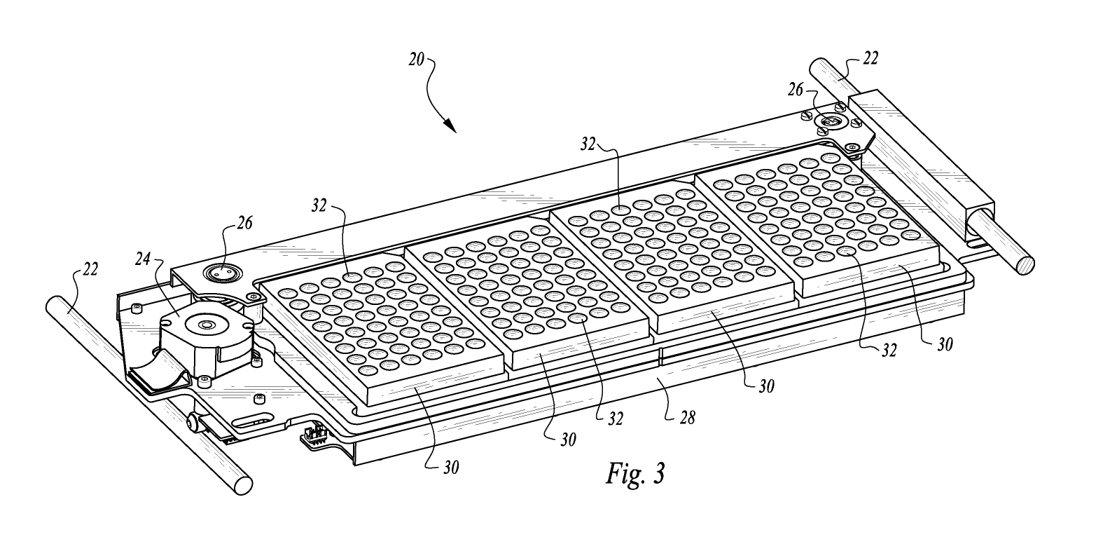

Typically, the shaker motor 24 will stop briefly during this dispensing process and then recommence shaking. Any movement of the shaker assembly 20 front to back (along arrow C of FIGS. 1 and 3) is sufficiently slow that it does not interrupt the shaking procedure for the specimen and reagent. This process can be continued potentially for as many tests as there are samples stored on the sample rack 12 and for the number of wells 32 available on the microtiter plates 30 on the shaker assembly 20.

After the predetermined amount of time for the assay has elapsed, the upper carriage 16 is moved appropriately to position the camera 40 over wells 32 for which the time has elapsed. The shaker assembly 20 is typically briefly stopped while a photograph is taken with the camera 40. Before this photograph is taken, the LED board 29 is energized so that light emitting from the LED board passes through the diffuser 27 and through the transparent or translucent microtiter plate 30 for backlighting of the photograph. The shaker assembly 20 can then recommence the shaking procedure.

An image file is created by the camera 40 and this image file is archived. The image file can also be transmitted to a display for viewing by a trained operator so that the photograph can be read to determine what the result of the test is. Alternatively, the reading of the test can be automated. Test results can be added to this archive data file.

When all of the wells 32 on all of the microtiter plates 30 have been read the microtiter plates 30 that have been fully utilized can be removed from the shaker assembly 20 and disposed of or washed and sanitized for reuse. New (or cleaned) microtiter plates 30 can be loaded onto the shaker assembly 20. Sample containers can be removed from the sample rack 12 and new samples loaded into the sample rack 12, and additional reagent can be provided into the reagent rack 14 and the entire testing procedure can continue with a new set of samples.

While the machine 10 and process are particularly defined herein for RPR tests such as a test for evaluating whether or not agglutination/flocculation has occurred when a sample is brought into contact with a reagent, other similar tests could also be performed utilizing the process and machine 10 of this invention. In particular, any tests which require combination of two or more liquids together, with or without the requirement of shaking and/or elapsed time, and which require a photograph to create an image of the liquids after any reaction has occurred, could be performed utilizing the machine 10 and process of this invention.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. A process for automated performance of a rapid plasma reagin test, including the steps of:
loading a sample into a first location;
loading an RPR reagent into a second location;
gathering a predefined amount of reagent and a predefined amount of sample with an automated aspirator and dispenser;
dispensing the reagent and the sample from the aspirator and dispenser into a well of an at least partially transparent plate;
automatically shaking the plate for a predetermined amount of time; and
photographing the well on the plate.

2. The process of claim 1 wherein said photographing step includes supplying back light up through the at least partially transparent plate into the well and photographing from above the plate.

3. The process of claim 1 wherein said gathering step occurs sequentially for the sample and the reagent.

4. The process of claim 3 wherein said dispensing step occurs together for the sample and the reagent.

5. The process of claim 1 wherein said gathering step includes positioning the plate on a shaker assembly and said shaking step includes powering a shaker motor on the shaker assembly for the predetermined amount of time.

6. The process of claim 1 wherein said photographing step includes moving a camera on a carriage to a position above the well to be photographed.

7. The process of claim 6 wherein said gathering step includes locating the aspirator and dispenser in the carriage.

8. A machine for performing a rapid plasma reagin test, comprising in combination:
an enclosure including a sample rack and reagent rack at a lower level thereof;
a shaker assembly located at a midlevel thereof;
an upper portion of the enclosure including an automated aspirator and dispenser movable to access locations on the sample rack, locations in the reagent rack and locations on a microtiter plate borne by the shaker assembly; and
a camera on an upper portion of the enclosure movable to be located over each of the locations of the microtiter plate.

9. The machine of claim 8 wherein said shaker assembly is capable of moving at least in a forwardly and rearwardly direction within the enclosure.

10. The machine of claim 8 wherein said aspirator and dispenser and said camera are located upon a common carriage.

11. The machine of claim 10 wherein said aspirator and dispenser is a syringe with a tip sized to fit into sample containers in said sample rack and to fit into a reagent container in said reagent rack.

12. The machine of claim 10 wherein said carriage moves both laterally and forwardly and rearwardly.

13. The machine of claim 11 wherein said shaker assembly includes a backlight beneath said microtiter plate.

14. A method for automated performance of an agglutination test, including the steps of:
loading a sample to be tested into a first location;
loading a reagent into a second location;
gathering a predefined amount of reagent and a predefined amount of sample with an automated aspirator and dispenser;
dispensing the reagent and the sample from the aspirator and dispenser into a well;
automatically shaking the well for a predetermined amount of time; and
detecting agglutination indicative of a positive reaction between the sample and the reagent.

15. The method of claim 14 wherein said detecting step includes photographing the well after said automatically shaking step.

16. The process of claim 15 wherein said photographing step includes moving a camera on a carriage to a position above the well to be photographed.

17. The process of claim 16 wherein said gathering step includes locating the aspirator and dispenser in the carriage.

18. The process of claim 15 wherein said photographing step includes supplying back light up through the the well which is at least partially transparent and photographing the well from above the well.

19. The process of claim 14 wherein said gathering step occurs sequentially for the sample and the reagent and said dispensing step occurs together for the sample and the reagent.

20. The process of claim 14 wherein said gathering step includes positioning the well on a shaker assembly and said shaking step includes powering a shaker motor on the shaker assembly for the predetermined amount of time.
